(19) United States
(12) Patent Application Publication
(10) Pub. No.: US 2010/0115661 A1
(43) Pub. Date: May 6, 2010

(54) METHODS AND COMPOSITIONS FOR THE MODULATION OF AMINO ACID BIOSYNTHESIS

(75) Inventors: John H. Duesing, Des Moines, IA (US); Anthony J. Kinney, Wilmington, DE (US); Daniel Siehl, Menlo Park, CA (US)

Correspondence Address: ALSTON & BIRD LLP;PIONEER HI-BRED INTERNATIONAL, INC., BANK OF AMERICA PLAZA, 101 SOUTH TRYON STREET, SUITE 4000, CHARLOTTE, NC, 28280-4000, US

(73) Assignees: Pioneer Hi-Bred International, Inc., Johnston, IA (US); E.I. du Pont de Nemours and Company, Wilmington, DE (US)

(21) Appl. No.: 12/581,331

(22) Filed: Oct. 19, 2009

Related U.S. Application Data
(60) Provisional application No. 61/109,550, filed on Oct. 30, 2008.

(51) Int. Cl. C12N 15/82; A01H 5/00

(52) U.S. Cl. 800/278; 800/298

(57) ABSTRACT

Compositions and methods for increasing the level of one or more selected free ε and/or α-N-acetylated amino acids in a selected tissue or organ of a plant are provided. In specific embodiments, the plant, plant part, seed or grain comprises a free α-N-acetylated amino acid content of at least 200 ppm or a ratio of free α-N-acetylated amino acid content to free non-α-N-acetylated amino acid content of about 3 to about 1000. Compositions comprising plants, plant parts, seed and grain having stably incorporated into their genome a heterologous polynucleotide encoding an amino acid-N-acetyltransferase polypeptide operably linked to a promoter active in the seed are provided. Further provided are compositions comprising a plant, plant part, seed or grain having stably incorporated into their genome a first heterologous polynucleotide encoding a first amino acid-N-acetyltransferase polypeptide operably linked to a first promoter active in the seed and a second heterologous polynucleotide encoding a second amino acid-N-acetyltransferase polypeptide operably linked to a second promoter active in the seed, wherein the first and the second amino acid-N-acetyltransferase polypeptide acetylate the α-amine of distinct amino acids. Compositions comprising food sources, feed and supplements, along with methods of increasing the nutritional value of a plant, plant part, seed or grain, are further provided.

### CROSS-REFERENCE TO RELATED APPLICATIONS

This application claims priority to U.S. Provisional Application No. 61/109,550, filed on Oct. 30, 2008 which is incorporated by reference in its entirety.

### FIELD OF THE INVENTION

This invention is in the field of molecular biology. More specifically, this invention pertains to modulating amino acid content in a plant and improving the nutritional value of a plant or plant part.

### BACKGROUND OF THE INVENTION

Human beings and livestock require eight essential amino acids in their diets. Diets based predominantly on a single cereal or legume species result in amino acid deficiencies due to the nutritional limitation of seed proteins that may have a negative effect(s) on the dietary needs of human beings and animals. For example, the proteins in cereal seeds are deficient in lysine and tryptophan, whereas legume seeds contain proteins deficient in the sulfur-containing amino acids, methionine and cysteine. The use of seed proteins in feed of livestock necessitates that the diet has a prescribed amino acid composition in order to promote the health of animals, efficient growth, and good quality of meat and milk. Therefore, it is advantageous to modify existing plant protein resources, in particular, for the composition of essential amino acids in order to be better adapted to the needs of a specified animal.

Efforts have been made to match the composition of vegetable amino acids to the dietary needs of humans and animals, but with limited success. The use of nutritionally superior plant mutants and tissues thereof is however compromised by negative pleiotropic effects. These problems include poor seed germination, slow dry-down, reduced yield, increased microbial and insect susceptibility, and poor milling characteristics. Accordingly, methods and compositions which increase the nutritional value of plants and parts thereof are needed.

### BRIEF SUMMARY OF THE INVENTION

Compositions and methods for increasing the level of one or more selected acetylated amino acid in a selected tissue or organ of a plant are provided. In specific embodiments, the plant, plant part, seed or grain comprises a free α-N-acetylated amino acid content of at least 200 ppm or a ratio of free α-N-acetylated amino acid content to free non-α-N-acetylated amino acid content of about 3 to 1000.

Further provided are compositions comprising plants, plant parts, seed and grain having stably incorporated into their genome a heterologous polynucleotide encoding an amino acid-N-acetyltransferase polypeptide operably linked to a promoter active in the seed of the plant. The seeds of such plants can comprise an increased level of free α-N-acetyl-methionine or free α- or ε-N-acetyl-lysine when compared to a control plant not expressing the heterologous polynucleotide; a free α-N-acetylated amino acid content of at least 200 ppm; or, a ratio of free α-N-acetylated amino acid content to free non-α-N-acetylated amino acid content of about 3 to 1000.

Further provided are compositions comprising a plant or a plant part having stably incorporated into its genome a first heterologous polynucleotide encoding a first amino acid-N-acetyltransferase polypeptide operably linked to a first promoter active in the seed and a second heterologous polynucleotide encoding a second amino acid-N-acetyltransferase polypeptide operably linked to a second promoter active in the seed, wherein the first and the second amino acid-N-acetyltransferase polypeptides acetylate distinct amino acids.

Compositions comprising food sources, feed and supplements, along with methods of increasing the nutritional value of seed or grain, are further provided.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments set forth herein will come to mind to one skilled in the art to which these embodiments pertain having the benefit of the teachings presented in the foregoing descriptions and the associated drawings. Therefore, it is to be understood that the embodiments are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

Compositions and methods for increasing the level of one or more selected ε- and/or α-N-acetylated amino acid in a selected tissue or organ of a plant are provided. Acetylation of the α-amine group of an amino acid provides for the metabolic sequestration of the acetylated amino acid from the free-amino acid pool. This sequestration of one or more selected free ε- and/or α-N-acetylated amino acids in a seed or a grain allows for an accumulation of amino acids without influencing pathways which regulate the levels of free amino acids, and thereby allows for an increase in the overall nutritional value of the seed or grain. The methods and compositions find use in the animal feed industry to produce feeds and nutritional supplements having elevated levels of free ε- and/or α-N-acetylated amino acids.

The methods and compositions of the invention provide for the acetylation of any amino acid or any combination thereof. The term “amino acid” refers generally to any of the amino acids that are known to occur in a biological system and includes glycine, alanine, valine, leucine, isoleucine, serine, threonine, cysteine, methionine, proline, aspartic acid, asparagine, glutamic acid, glutamine, lysine, arginine, histidine, phenylalanine, tyrosine, and tryptophan or derivative and analogs thereof. For details on amino acid nomenclature, see, for example, The IUPAC-IUB Joint-Commission-On-Biochemical-Nomenclature (JCBN) Nomenclature And Symbolism For Amino-Acids And Peptides-Recommendations 1983 by A. Cornishbowden in the Biochemical Journal, 1984, Vol. 219, No. 2, pages: 345-373.

As used herein, the term “essential amino acid” refers to an amino acid that cannot be synthesized de novo by an organism and therefore must be supplied to the organism in the diet. Essential amino acids for humans include isoleucine, leucine, lysine, methionine, phenylalanine, threonine, tryptophan, and valine. For humans, the amino acids arginine, cysteine, glycine, glutamine and tyrosine are considered conditionally essential, meaning that they are not normally required in the diet, but must be supplied exogenously to specific populations that do not synthesize adequate amounts. One of skill will recognize that which amino acids are essential will vary from species to species, as different metabolisms are able to synthesize different substances.

The net protein utilization of an organism is affected by the limiting essential amino acid content (the essential amino acid found in the smallest quantity in the foodstuff), and thus, while the level of any free ε- and/or α-N-acetylated amino acid can be increased by the methods and compositions disclosed herein, in specific embodiments, the selected free ε- and/or α-N-acetylated amino acid which is increased or the combination of selected free ε- and/or α-N-acetylated amino acids which are increased in the plant, plant part, grain or seed comprises at least the amino acid moiety that represents the limited essential amino acid of that plant. In specific embodiments, the level of free α-N-acetyl-methionine, free α-N-acetyl-cysteine, free α-N-acetyl-lysine, free E-N-acetyl-lysine, free α-N-acetyl-tryptophan, free α-N-acetyl-threonine or any combination thereof is elevated in the plant, plant part, seed or grain. In other embodiments, in wheat or rice, at least the level of free α-N-acetyl-lysine and/or free ε-N-acetyl-lysine is increased; in maize, at least the level of free ε- and/or α-N-acetyl-lysine and/or free α-N-acetyl-tryptophan is increased; in soybean, at least the levels of free α-N-acetyl-cysteine, free α-N-acetyl-methionine, free ε- and/or α-N-acetyl-lysine, and/or free α-N-acetyl-tryptophan is increased; and in legumes, at least the level of free α-N-acetyl-methionine and/or free α-N-acetyl-cysteine is increased. Other combinations of desirable alterations in free α-N-acetylated amino acid content are discussed elsewhere herein.

The term “free amino acid” or “free amino acid pool” refers to the amino acids which are not covalently bonded to another amino acid.

The term “acetylated amino acid” refers to any amino acid comprising an acetyl group. The term “α-N-acetylated amino acid” or “α-N-acetyl-amino acid” refers to an amino acid comprising an acetyl group on the α-amino group of the amino acid. As shown below, R represents the side chain and the α-amine is denoted with an asterisk.

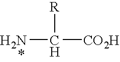

Other forms of acetylated amino acids include the addition of an amino group on the epsilon carbon of lysine. Such amino acids are referred to herein as “ε-N-acetylated-amino acids.”

Thus, methods of increasing the level in a plant, plant part, seed or grain of any α-N-acetylated amino acid are provided including, but are not limited to, α-N-acetyl-methionine, α-N-acetyl-cysteine, α-N-acetyl-lysine, ε-N-acetyl-lysine, α-N-acetyl-tryptophan, α-N-acetyl-threonine or any combination thereof. In further embodiments, the level of at least one of α-N-acetyl-glycine, α-N-acetyl-alanine, α-N-acetyl-valine, α-N-acetyl-leucine, α-N-acetyl-isoleucine, α-N-acetyl-serine, α-N-acetyl-proline, α-N-acetyl-aspartic acid, α-N-acetyl-asparagine, α-N-acetyl-glutamic acid, α-N-acetyl-glutamine, α-N-acetyl-arginine, α-N-acetyl-histidine, α-N-acetyl-phenylalanine, and/or α-N-acetyl-tyrosine or any combination thereof is increased.

In another embodiment, the term “free acetylated amino acid level” refers to the total amount of the free acetylated amino acid in a whole plant, plant part, plant tissue (seed, kernel, or grain) or plant cell. “Modulating the free ε- and/or α-N-acetylated amino acid content” includes any decrease or increase in the total free ε- and/or α-N-acetylated amino acid level in a whole plant, plant part (seed, grain, or kernel), plant tissue, or plant cell. For example, modulating the free ε- and/or α-N-acetylated amino acid content can comprise either an increase or a decrease in overall free ε- and/or α-N-acetylated amino acid level of about 0.1%, 0.5%, 1%, 5%, 10%, 15%, 20%, 25%, 30%, 35%, 40%, 45%, 50%, 55%, 60%, 65%, 70%, 75% 80%, 85% 90%, 95% 100%, 120% or greater when compared to a control plant or plant part. Alternatively, modulating the free ε- and/or α-N-acetylated amino acid content can comprise either an increase or a decrease in overall free ε- and/or α-N-acetylated amino acid level of about 0.1% to about 1%; about 1% to about 5%; about 10% to about 15%; about 15% to about 20%; about 20% to about 30%; about 30% to about 40%; about 40% to about 50%; about 50% to about 60%; about 60% to about 70%; about 70% to about 80%; about 80% to about 90%; about 90% to about 100%; about 100% to about 120% or greater when compared to a control plant or plant part.

In still other embodiments, the level of at least one or more free ε- and/or α-N-acetylated amino acid comprises at least about 200 ppm to about 4000 ppm or about 100 ppm to about 10000 ppm in the plant, plant part, seed or grain. In other embodiments, the level of at least one or more free ε- and/or α-N-acetylated amino acid comprises about 100 ppm to about 150 ppm; about 150 ppm to about 200 ppm, about 200 ppm to about 250 ppm, about 250 ppm to about 300 ppm, about 300 ppm to about 350 ppm; about 350 pm to about 500 ppm, about 500 ppm to about 600 ppm, about 600 ppm to about 700 ppm, about 700 ppm to about 800 ppm, about 800 ppm to 1000 ppm, about 1000 ppm to about 1200 ppm, about 1200 ppm to about 1400 ppm, about 1400 ppm to about 1600 ppm, about 1600 ppm to about 1800 ppm, about 2000 ppm to about 2200 ppm, about 2200 ppm to about 2400 ppm, about 2400 ppm to about 2600 ppm, about 2600 ppm to about 2800 ppm, about 2800 ppm to about 3000 ppm, about 3000 pmm to about 3200 ppm, about 3200 ppm to about 3400 ppm, about 3400 ppm to about 3600 ppm, about 3600 ppm to about 3800 ppm, about 3800 ppm to about 4000 ppm, about 4000 ppm to about 4200 ppm, about 4200 ppm to about 4400 ppm, about 4400 ppm to about 4600 ppm or greater. In other embodiments, the level of at least one or more free α-N-acetylated amino acid comprises at least about 100 ppm, at least about 500 ppm, at least about 1000 ppm, at least about 1500 ppm, at least about 2000 ppm, at least about 2500 ppm, at least about 3000 ppm, at least about 4000 ppm or greater.

For example, the ratio of free ε- and/or α-N-acetylated amino acid to free non-ε- and/or α-N-acetylated amino acid of one or more specific amino acid(s) could be altered and thereby modulate the free ε- and/or α-N-acetylated amino acid content of the plant or plant part when compared to a control plant.

In specific embodiments, the free α-N-acetyl-amino acids to free non-α-N-acetylated amino acid ratio of at least about 1:1 to about 1000:1 or greater; about 3:1 to about 1000:1, about 1:1, about 0.5:1 to about 100:1, about 0.5:1 to about 20:1, about 1:1 to about 500:1, about 500:1 to about 800:1, about 800:1 to about 1000:1, or about 900:1 to about 1100.

In other embodiments, the ratio of free α-N-acetyl-methionine to free non-α-N-acetylated methionine is increased, the ratio of free α-N-acetyl-lysine to free non-α-N-acetylated lysine is increased, the ratio of free ε-N-acetyl-lysine to free non-ε-N-acetyl-lysine is increased, the ratio of free α-N-acetyl-threonine to free non-α-N-acetylated threonine is increased, the ratio of free α-N-acetyl-cysteine to free non-α-N-acetylated cysteine is increased, and/or the ratio of free α-N-acetyl-tryptophan to free non-α-N-acetylated tryptophan is increased. In specific embodiments, the free α-N-acetyl-methionine to free non-α-N-acetylated methionine ratio of at least about 100:1 to 1000:1 or greater, about 20:1, about 10:1 to about 1000:1, about 10:1 to about 50:1, about 50:1 to about 500:1, about 500:1 to about 1000:1; and/or the free α-N-acetyl-lysine to free non-α-N-acetylated lysine ratio is at least about 20:1 to about 200:1 or greater; at least about 185:1, about 150:1 to about 190:1 or at least about 75:1 to about 125:1; and/or the free α-N-acetyl-threonine to free non-α-N-acetylated threonine ratio is at least about 10:1 to 120:1 or greater; about 124:1, about 10:1 to about 50:1, about 50:1 to about 100:1, about 100:1 to about 125:1, about 120:1 to about 130:1; and/or the free α-N-acetyl-tryptophan to free non-α-N-acetylated tryptophan ratio is at least about 1.2:1 to about 3:1 or greater, about 2.61:1 or about 2:1 to about 2.5:1, or about 1.7 to about 2.8; and/or, the free α-N-acetyl-cysteine to free non-α-N-acetylated cysteine ratio is at least about 100:1 to about 1000:1 or greater, about 1049:1, about 100:1 to about 500:1, about 500:1 to about 1000:1, about 900:1 to about 1050:1, about 1025:1 to about 1055:1. See, also Takahashi et al. (2003) Planta 217:577-586 (herein incorporated by referenced) for further information regarding levels of free α-N-acetylated amino acids.

Methods for assaying for a modulation in the free ε- and/or α-N-acetylated amino acid content are known in the art. For example, the total free ε- and/or α-N-acetylated amino acid content of the plant, plant part, plant cell, seed or grain can be measured. Alternatively, the total free ε- and/or α-N-acetylated amino acid content in the embryo of the seed or grain can be measured. Representative methods to measure free ε- and/or α-N-acetylated amino acid content include the combination of high performance liquid chromatography and mass spectrometry. Each of these references is herein incorporated by reference. Alonso et al. (1991) Neurochem. Res. 16:787-794; Moffett et al. (2007) Prog. Neurobiol. 81: 89-131; Baena et al. (2005) Electrophoresis 26: 2622-2636; Alonso et al. (1985) Anal. Biochem. 146: 252-259; Tavazzi et al. (2000) Anal. Biochem. 277: 104-108; Tavazzi et al. (2005) Clin. Biochem. 38, 997-1008; Tranberg et al. (2005) Anal. Biochem. 343: 179-182; Faull et al. (1999) Neurochem. Res. 24: 1249-1261; Gerlo et al. (2006) Anal. Chim. Acta 571: 191-199; Roessner et al. (2006) Plant Physiol. 142, 1087-1101; Jacobs et al. (2007) Metabolomics 3: 307-317; Al-Dirbashi et al. (2007) Biomed. Chromatogr. 21: 898-902; each of which is incorporated by reference in their entirety.

In specific embodiments, the methods and compositions increase the level of free α-N-acetylated amino acids but do not increase, or increase to a minimal extend, the acetylation of N-terminal amines of proteins and/or the acetylation of lysine-epsilon amines of proteins. In specific embodiments, the minimal increases in these forms of acetylation comprise any increase that does not negatively impact the agronomical performance of the plant, plant part, seed or grain. By devising appropriate screening methods, it is possible to differentiate naturally occurring N-acetyltransferases that acetylate only free amino acids from those that acetylate N-terminal amines and lysine-epsilon amines of proteins. Further refinement of substrate specificity can be achieved through methods of enzyme optimization such as DNA shuffling.

The “targeted accumulation” of a selected free ε- and/or α-N-acetylated amino acid means that the free ε- and/or α-N-acetylated amino acid accumulates in a selected tissue or organ of the plant. In specific embodiments, the selected free ε- and/or α-N-acetylated amino acid accumulates in the seed or the grain of the plant. In specific embodiments, the free α-N-acetylated amino acids do not accumulate in the seed coat of the seed.

### II. N-Acetyltransferase Polypeptides

While any method can be employed to increase the level of one or more selected free ε- and/or α-N-acetylated amino acids, in one embodiment, the level of the selected free ε- and/or α-N-acetylated amino acid is increased by increasing the level of activity of an N-acetyltransferase polypeptide in the seed of the plant. As used herein the term “N-acetyltransferase polypeptide” or “a polypeptide having N-acetyltransferase activity” refers to a polypeptide having the ability to transfer an acetyl group to a substrate of interest. As referred to herein, an “amino acid-α-N-acetyltransferase” polypeptide or a “polypeptide having amino acid-α-N-acetyltransferase activity” comprises a polypeptide having the ability to transfer an acetyl group onto the α-amino group of a selected amino acid. In specific embodiments, the amino acid α-N-acetyltransferase polypeptide can further acetylate the ε-amino acid of lysine.

Amino acid-N-acetyltransferase polypeptides are known in the art. See, for example, table 1 which provides non-limiting examples of amino acid-N-acetyltransferases and their amino acid substrate. Each of the references cited in Table 1 is hereby incorporated by reference in their entirety.

TABLE 1

| | | Amino acid |
|---|---|---|
| N-acetyltransferase | Reference | substrate |
| Glyphosate-N- | WO 200501215 | L-aspartate |
| acetyltransferase | | L-serine |
| | | L-histidine |
| | | L-tyrosine |
| | | L-threonine |
| | | L-valine |
| | | L-glutamine |
| | | L-asparagine |
| | | L-alanine |
| | | L-glycine |
| | | L-cysteine |
| N2- | Slocum et al. (2005) Science | L-glutamate |
| acetylornithine:glutamate | 43: 729-745 | |
| acetyltransferase | | |
| (NAOGAcT) (EC2.3.1.35) | | |
| ArgA | Errey et al. (2005) Journal of | L-glutamate |
| | Bacteriology 187: 3039-3044 | |

Several families of N-acetyltransferase polypeptides are known. Such families include the PCAF/GCN5 family, the p300/CBP family, the TAF250 family, the SRC1 family and the MOZ family. See, for example, Kouzarides et al. (2002) The EMBO J. 19:1176-1179 and Kouzarides (1999) Current Opinions in Genetics Development 79:40-48, both of which are herein incorporated by reference. Additional N-acetyltransferases include members of the N-terminal acetyltransferases (NAT) family. Such members include NatA, NatB, and NatC, which contain Ard1p, Nat3p and Mak3p catalytic subunits. See, for example, Polevoda et al. (2003) J. Mol. Biol. 325:595-622, herein incorporated by reference. The N-terminal acetyltransferase family has been characterized as comprising at least 6 protein families including Ard1p, Nat3p, and Mak3p, which correspond to the catalytic subunits of the yeast N-terminal acetyltransferases described above. Additional families include the CAM family, the BAA family, and the Nat5p family. See, Polevoda et al. (2003) J. Mol. Biol. 325:595-622 for a sequence alignment of various members of the N-terminal acetyltransferase family. Another family of N-acetyltransferases includes the GCN5-related N-acetyltransferases. See, INTERPRO Acc. No. IPR000182, PFAM Accession No. PF00583 and Prosite profile PS51186. Members of this family include glyphosate-N-acetyltransferase polypeptides (WO 200501215) and N-acetylglutmine (NAGS) polypeptides (Errey et al. (2005) Journal of Bacteriology 187:3039-3044).

Biologically active fragments and variants of the amino acid-α-N-acetyltransferase polypeptides will continue to retain activity, i.e., acetylate the α-amine of at least one or more free amino acid and/or the ε-amino acid of lysine. Methods are described elsewhere herein for assaying for amino acid acetylation. For example, the ability of an amino acid-N-acetyl transferase polypeptide to acetylate amino acids can be determined using an indirect assay in which acetylation of amino acids is inferred by detecting free coenzyme A with the sulfhydryl reagent 5,5′-dithio-bis(2-nitrobenzoate) (DTNB). In such exemplary assays, the enzyme can be present at 0.1 μM and amino acids at 10 mM. KCl can provided at 100 mM to simulate physiological ionic strength. Under these conditions, acetylation of various amino acids can be detected. It is recognized KCL concentration can be altered to allow for the acetylation of certain amino acids.

In one embodiment, the level of an amino acid-N-acetyltransferase is increased in the seed of a plant, wherein the amino acid-N-acetyltransferase acetylates the α-amine of a selected free amino acid or the ε-amine of free lysine and/or alternatively, has the ability to acetylate the α-amine of a distinct set of selected amino acids. Methods for optimization of such activity are discussed elsewhere herein. In other embodiments, the level of a first amino acid-N-acetyltransferase having the ability to acetylate at least an ε- or α-amino of a free selected amino acid is increased and the level of a second and distinct amino acid-N-acetyltransferase having the ability to acetylate an ε- and/or α-amine of at least a second distinct amino acid is increased in a single plant, plant part, grain or seed. In this manner, one can customize the specific ε- and/or α-N-acetylated amino acid profile of the plant seed and plant material derived there from.

In one embodiment, a glyphosate-N-acetyltransferase polypeptide is optimized to acetylate the ε- and/or α-amine of an amino acid(s) of interest. Methods for the optimization of this activity are discloses elsewhere herein. In specific embodiments, the optimization of a glyphosate-N-acetyltransferase (GLYAT) polypeptide to acetylate the ε- and/or α-amine of an amino acid(s) of interest is carried out under conditions that do not require GLYAT polypeptide to retain the ability to acetylate glyphosate or a derivative thereof. In other embodiments, the optimization of GLYAT is carried out under conditions that allow for the GLYAT enzyme to retain the ability to acetylate glyphosate and further acetylate the ε- and/or α-amine of one or more free amino acid of interest. In non-limiting examples, the GLYAT polypeptide is optimized to acetylate the α-amine of free methionine, cysteine, tryptophane, threonine, or lysine.

In further embodiments, the amino acid-N-acetyltransferase polypeptide acetylates the α-amino group of one or more selected amino acid and further the amino acid-N-acetyltransferase is not able to acetylate the N-terminal amines of proteins and/or acetylate the lysine-epsilon amines of proteins and/or acetylates these moieties to a minimal extent (i.e., does not negatively impact the agronomic characteristics of the plant, plant part, seed, or grain).

In further embodiments the acetylated amino acid pool may be further enhanced by increasing the supply of free amino acids in the plant, using methods known in the art (Bartlem et al. (2000) Plant Phsiology 123: 101-110; Zeh et al. (2001) Plant Phsysiol. 127:792-802; Amir et al. (2002) Trends Plant Sci 7:153-156; Sirko et al. (2005) J. Exp. Botany 55:1881-1888; and, Lee et al. (2005) Plant Journal 41: 685-696; each of which is herein incorporated by reference.

a. Methods to Optimize Amino Acid-N-Acetyltransferase Activity

N-acetyltransferase polypeptides can be used as substrates for a variety of diversity generating procedures, e.g., mutation, recombination and recursive recombination reactions, to produce amino acid-α-N-acetyltransferase polynucleotides. A variety of diversity generating protocols can be used to allow for the selection of the desired activity (i.e., the acetylation of α-amine of one or more selected free amino acid.) Such procedures provide robust, widely applicable ways of generating diversified polynucleotides and sets of polynucleotides (including, e.g., polynucleotide libraries) useful, e.g., for the engineering or rapid evolution of polynucleotides, proteins, pathways, cells and/or organisms with new and/or improved characteristics. The process of altering the sequence can result in, for example, single nucleotide substitutions, multiple nucleotide substitutions, and insertion or deletion of regions of the nucleic acid sequence.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity generating procedures described herein can be the generation of one or more polynucleotides, which can be selected or screened for polynucleotides that encode proteins with or which confer desirable properties. Following diversification by one or more of the methods described herein, or otherwise available to one of skill, any polynucleotides that are produced can be selected for a desired activity or property, e.g. altered Kₘ for one or more selected amino acid of interest, altered Kₘ for acetyl CoA, use of alternative cofactors (e.g., propionyl CoA), increased k_{cat}, etc. This can include identifying any activity that can be detected, for example, in an automated or automatable format, by any of the assays in the art. For example, amino acid-α-N-acetyltransferase homologs with increased specific activity can be detected by assaying the conversion of the amino acid of interest to the α-N-acetyl form, e.g., by mass spectrometry. Additional details regarding recombination and enzymatic activity of interest can be found, e.g., in U.S. Pub. No. 2002/0058249.

Descriptions of a variety of diversity generating procedures, including multigene shuffling and methods for generating modified nucleic acid sequences encoding multiple enzymatic domains, are found the following publications and the references cited therein: Soong et al. (2000) Nat. Genet. 25(4): 436-39; Stemmer et al. (1999) Tumor Targeting 4: 1-4; Ness et al. (1999) Nature Biotech. 17:893-896; Chang et al. (1999) Nature Biotech. 17: 793-797; Minshull (1999) Current Opinion in Chemical Biology 3: 284-290; Christians et al. (1999) Nature Biotech. 17: 259-264; Crameri et al. (1998) Nature 391: 288-291; Crameri et al. (1997) Nature Biotech. 15: 436-438; Zhang et al. (1997) Proc. Nat'l. Acad. Sci. USA 94: 4504-4509; Patten et al. (1997) Current Opinion in Biotech. 8: 724-733; Crameri et al. (1996) Nature Med. 2:100-103; Crameri et al. (1996) Nature Biotech. 14:315-319; Gates et al. (1996) J. Mol. Biol. 255: 373-386; Stemmer (1996) “Sexual PCR and Assembly PCR” in The Encyclopedia of Molecular Biology (VCH Publishers, New York) pp. 447-457; Crameri and Stemmer (1995) BioTechniques 18: 194-195; Stemmer et al., (1995) Gene 164: 49-53; Stemmer (1995) Science 270: 1510; Stemmer (1995) Bio/Technology 13: 549-553; Stemmer (1994) Nature 370: 389-391; and Stemmer (1994) Proc. Nat'l. Acad. Sci. USA 91:10747-10751.

Mutational methods of generating diversity include, for example, site-directed mutagenesis (Ling et al. (1997) Anal Biochem. 254(2): 157-178; Dale et al. (1996) Methods Mol. Biol. 57:369-374; Smith (1985) Ann. Rev. Genet. 19:423-462; Botstein (1985) Science 229:1193-1201; Carter (1986) Biochem. J. 237:1-7; and Kunkel (1987) Nucleic Acids & Molecular Biology (Eckstein, F. and Lilley, D. M. J. eds., Springer Verlag, Berlin)); mutagenesis using uracil containing templates (Kunkel (1985) Proc. Natl. Acad. Sci. USA 82:488-492; Kunkel et al. (1987) Methods in Enzymol. 154, 367-382; and Bass et al. (1988) Science 242:240-245); oligonucleotide-directed mutagenesis (Methods in Enzymol. 100: 468-500 (1983); Methods in Enzymol. 154: 329-350 (1987); Zoller & Smith (1982) Nucleic Acids Res. 10:6487-6500; Zoller & Smith (1983) Methods in Enzymol. 100:468-500; and Zoller & Smith (1987) Methods in Enzymol. 154:329-350); phosphorothioate-modified DNA mutagenesis (Taylor et al. (1985) Nucl. Acids Res. 13: 8749-8764; Taylor et al. (1985) Nucl. Acids Res. 13: 8765-8787; Nakamaye & Eckstein (1986) Nucl. Acids Res. 14: 9679-9698; Sayers et al. (1988) Nucl. Acids Res. 16:791-802; and Sayers et al. (1988) Nucl. Acids Res. 16: 803-814); mutagenesis using gapped duplex DNA (Kramer et al. (1984) Nucl. Acids Res. 12: 9441-9456; Kramer (1987) Methods in Enzymol. 154:350-367; Kramer et al. (1988) Nucl. Acids Res. 16: 7207; and Fritz et al. (1988) Nucl. Acids Res. 16: 6987-6999).

Additional suitable methods include point mismatch repair (Kramer et al. (1984) Cell 38:879-887), mutagenesis using repair-deficient host strains (Carter et al. (1985) Nucl. Acids Res. 13: 4431-4443; and Carter (1987) Methods in Enzymol. 154: 382-403), deletion mutagenesis (Eghtedarzadeh (1986) Nucl. Acids Res. 14: 5115), restriction-selection and restriction-purification (Wells et al. (1986) Phil. Trans. R. Soc. Lond. A 317: 415-423), mutagenesis by total gene synthesis (Nambiar et al. (1984) Science 223: 1299-1301; Sakamar (1988) Nucl. Acids Res. 14: 6361-6372; Wells et al. (1985) Gene 34:315-323; and Grundstrom et al. (1985) Nucl. Acids Res. 13: 3305-3316); double-strand break repair (Mandecki (1986); Arnold (1993) Current Opinion in Biotechnology 4:450-455; and Proc. Natl. Acad. Sci. USA, 83:7177-7181). Additional details on many of the above methods can be found in Methods in Enzymology Volume 154, which also describes useful controls for trouble-shooting problems with various mutagenesis methods.

Additional details regarding various diversity generating methods can be found in the following U.S. patents, PCT publications, and EPO publications: U.S. Pat. No. 5,605,793, U.S. Pat. No. 5,811,238, U.S. Pat. No. 5,830,721, U.S. Pat. No. 5,834,252, U.S. Pat. No. 5,837,458, WO 95/22625, WO 96/33207, WO 97/20078, WO 97/35966, WO 99/41402, WO 99/41383, WO 99/41369, WO 99/41368, EP 752008, EP 0932670, WO 99/23107, WO 99/21979, WO 98/31837, WO 98/27230, WO 98/13487, WO 00/00632, WO 98/42832, WO 99/29902, WO 98/41653, WO 98/41622, WO 98/42727, WO 00/18906, WO 00/04190, WO 00/42561, WO 00/42559, WO 00/42560, WO 01/23401, and WO 01/64864. Certain U.S. applications provide additional details regarding various diversity generating methods, including U.S. Ser. No. 09/407,800; U.S. Ser. No. 09/166,188, U.S. Pat. No. 6,379,964; U.S. Pat. No. 6,376,246; WO 00/42561; U.S. Pat. No. 6,436,675; WO 00/42560); U.S. Ser. No. 09/618,579; WO 00/42559; and, U.S. Ser. No. 60/186,482.

In brief, several different general classes of sequence modification methods, such as mutation, recombination, etc. are applicable and set forth in the references above. That is, alterations to the component nucleic acid sequences to produced modified gene fusion constructs can be performed by any number of the protocols described, either before cojoining of the sequences, or after the cojoining step. The following exemplify some of the different types of preferred formats for diversity generation in the context of the present invention, including, e.g., certain recombination based diversity generation formats.

Nucleic acids can be recombined in vitro by any of a variety of techniques discussed in the references above, including e.g., DNAse digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process and many process variants is described in several of the references above, e.g., in Stemmer (1994) Proc. Nat'l. Acad. Sci. USA 91:10747-10751.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above.

Whole genome recombination methods can also be used in which whole genomes of cells or other organisms are recombined, optionally including spiking of the genomic recombination mixtures with desired library components. These methods have many applications, including those in which the identity of a target gene is not known. Details on such methods are found, e.g., in WO 98/31837 and WO 00/04190. Thus, any of these processes and techniques for recombination, recursive recombination, and whole genome recombination, alone or in combination, can be used to generate the modified amino acid-α-N-acetyltransferase sequences.

Synthetic recombination methods can also be used, in which oligonucleotides corresponding to targets of interest are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Details regarding such approaches are found in the references noted above, including, e.g., WO 00/42561, WO 01/23401, WO 00/42560, and, WO 00/42559.

In silico methods of recombination can be affected in which genetic algorithms are used in a computer to recombine sequence strings which correspond to homologous (or even non-homologous) nucleic acids. The resulting recombined sequence strings are optionally converted into nucleic acids by synthesis of nucleic acids which correspond to the recombined sequences, e.g., in concert with oligonucleotide synthesis gene reassembly techniques. This approach can generate random, partially random or designed variants. Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in WO 00/42560 and WO 00/42559. Extensive details regarding in silico recombination methods are found in these applications.

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library(ies) is annealed with partial, or, often approximately full length ssDNA or RNA corresponding to the opposite strand. Assembly of complex chimeric genes from this population is then mediated by nuclease-base removal of non-hybridizing fragment ends, polymerization to fill gaps between such fragments and subsequent single stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in WO 01/64864.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library of enriched sequences which hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids.

Mutagenesis employing polynucleotide chain termination methods have also been proposed (see e.g., U.S. Pat. No. 5,965,408 and can be applied to the present invention. In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombinational procedure termed “incremental truncation for the creation of hybrid enzymes” (“ITCHY”) described in Ostermeier et al. (1999) Nature Biotech 17:1205. This approach can be used to generate an initial library of variants which can optionally serve as a substrate for one or more in vitro or in vivo recombination methods. See, also, Ostermeier et al. (1999) Proc. Natl. Acad. Sci. USA 96: 3562-67; and Ostermeier et al. (1999) Biological and Medicinal Chemistry 7: 2139-44.

Mutational methods which result in the alteration of individual nucleotides or groups of contiguous or non-contiguous nucleotides can be favorably employed to introduce nucleotide diversity into the nucleic acid sequences and/or gene fusion constructs of the present invention. Many mutagenesis methods are found in the above-cited references. For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2: 28-33. Similarly, assembly PCR can be used, in a process which involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson et al. (1988) Science 241:53-57. Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin et al. (1992) Proc. Nat'l. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are found in Delegrave et al. (1993) Biotech. Res. 11:1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above.

Other procedures for introducing diversity into a genome, e.g. a bacterial, fungal, animal or plant genome can be used in conjunction with the above described and/or referenced methods. For example, in addition to the methods above, techniques have been proposed which produce nucleic acid multimers suitable for transformation into a variety of species (see, e.g., Schellenberger U.S. Pat. No. 5,756,316 and the references above). Transformation of a suitable host with such multimers, consisting of genes that are divergent with respect to one another, (e.g., derived from natural diversity or through application of site directed mutagenesis, error prone PCR, passage through mutagenic bacterial strains, and the like), provides a source of nucleic acid diversity for DNA diversification, e.g., by an in vivo recombination process as indicated above.

Alternatively, a multiplicity of monomeric polynucleotides sharing regions of partial sequence similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acids can be recovered by standard techniques, e.g., PCR and/or cloning, and recombined in any of the recombination formats, including recursive recombination formats, described above.

Methods for generating multispecies expression libraries have been described (in addition to the references noted above, see, e.g., U.S. Pat. No. 5,783,431, U.S. Pat. No. 5,824,485, and, U.S. Pat. No. 5,958,672. Multispecies expression libraries include, in general, libraries comprising cDNA or genomic sequences from a plurality of species or strains, operably linked to appropriate regulatory sequences, in an expression cassette. The cDNA and/or genomic sequences are optionally randomly ligated to further enhance diversity. The vector can be a shuttle vector suitable for transformation and expression in more than one species of host organism, e.g., bacterial species or eukaryotic cells. In some cases, the library is biased by preselecting sequences which encode a protein of interest, or which hybridize to a nucleic acid of interest. Any such libraries can be provided as substrates for any of the methods herein described.

The above described procedures have been largely directed to increasing nucleic acid and/or encoded protein diversity. However, in many cases, not all of the diversity is useful, e.g., functional, and contributes merely to increasing the background of variants that must be screened or selected to identify the few favorable variants. In some applications, it is desirable to preselect or prescreen libraries (e.g., an amplified library, a genomic library, a cDNA library, a normalized library, etc.) or other substrate nucleic acids prior to diversification, e.g., by recombination-based mutagenesis procedures, or to otherwise bias the substrates towards nucleic acids that encode functional products. For example, in the case of antibody engineering, it is possible to bias the diversity generating process toward antibodies with functional antigen binding sites by taking advantage of in vivo recombination events prior to manipulation by any of the described methods. For example, recombined CDRs derived from B cell cDNA libraries can be amplified and assembled into framework regions (e.g., Jirholt et al. (1998) Gene 215: 471) prior to diversifying according to any of the methods described herein.

Libraries can be biased towards nucleic acids which encode proteins with desirable enzyme activities. For example, after identifying a clone from a library which exhibits a specified activity, the clone can be mutagenized using any known method for introducing DNA alterations. A library comprising the mutagenized homologues is then screened for a desired activity, which can be the same as or different from the initially specified activity. An example of such a procedure is proposed in U.S. Pat. No. 5,939,250. Desired activities can be identified by any method known in the art. For example, WO 99/10539 proposes that gene libraries can be screened by combining extracts from the gene library with components obtained from metabolically rich cells and identifying combinations which exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids which have specified characteristics, e.g., hybridization to a selected nucleic acid probe. For example, WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences. In particular, single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived there from. Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described above.

“Non-stochastic” methods of generating nucleic acids and polypeptides are described in WO 00/46344. These methods, including proposed non-stochastic polynucleotide reassembly and site-saturation mutagenesis methods can be applied to the present invention as well. Random or semi-random mutagenesis using doped or degenerate oligonucleotides is also described in, e.g., Arkin et al. (1992) Biotechnology 10:297-300; Reidhaar-Olson et al. (1991) Methods Enzymol. 208:564-86; Lim and Sauer (1991) J. Mol. Biol. 219:359-76; Breyer and Sauer (1989) J. Biol. Chem. 264:13355-60; U.S. Pat. Nos. 5,830,650 and 5,798,208, and EP Patent 0527809 B1.

It will be readily appreciated that any of the above described techniques suitable for enriching a library prior to diversification can also be used to screen the products, or libraries of products, produced by the diversity generating methods. Any of the above described methods can be practiced recursively or in combination to alter nucleic acids, e.g., amino acid-N-acetyltransferase encoding polynucleotides.

Kits for mutagenesis, library construction and other diversity generation methods are also commercially available. For example, kits are available from, e.g., Stratagene (e.g., QuickChange™ site-directed mutagenesis kit; and Chameleon™ double-stranded, site-directed mutagenesis kit); Bio/Can Scientific, Bio-Rad (e.g., using the Kunkel method described above); Boehringer Mannheim Corp.; Clonetech Laboratories; DNA Technologies; Epicentre Technologies (e.g., 5 prime 3 prime kit); Genpak Inc.; Lemargo Inc.; Life Technologies (Gibco BRL); New England Biolabs; Pharmacia Biotech; Promega Corp.; Quantum Biotechnologies; Amersham International plc (e.g., using the Eckstein method above); and Anglian Biotechnology Ltd (e.g., using the Carter/Winter method above).

The above references provide many mutational formats, including recombination, recursive recombination, recursive mutation and combinations of recombination with other forms of mutagenesis, as well as many modifications of these formats. Regardless of the diversity generation format that is used, the nucleic acids of the present invention can be recombined (with each other, or with related (or even unrelated) sequences) to produce a diverse set of recombinant nucleic acids for use in the gene fusion constructs and modified gene fusion constructs of the present invention, including, e.g., sets of homologous nucleic acids, as well as corresponding polypeptides.

Many of the above-described methodologies for generating modified polynucleotides generate a large number of diverse variants of a parental sequence or sequences. In some preferred embodiments of the invention the modification technique (e.g., some form of shuffling) is used to generate a library of variants that is then screened for a modified polynucleotide or pool of modified polynucleotides encoding some desired functional attribute, e.g., improved amino acid-N-acetyltransferase activity. Exemplary enzymatic activities that can be screened for include catalytic rates (conventionally characterized in terms of kinetic constants such as k_{cat} and K_{M}), substrate specificity, and susceptibility to activation or inhibition by substrate, product or other molecules (e.g., inhibitors or activators).

In some embodiments of the invention, mass spectrometry is used to detect the acetylation of the amino acid(s) of interest.

For convenience and high throughput it will often be desirable to screen/select for desired modified nucleic acids in a microorganism, e.g., a bacteria such as E. coli. On the other hand, screening in plant cells or plants can in some cases be preferable where the ultimate aim is to generate a modified nucleic acid for expression in a plant system.

In some preferred embodiments, throughput is increased by screening pools of host cells expressing different modified nucleic acids, either alone or as part of a gene fusion construct. Any pools showing significant activity can be deconvoluted to identify single clones expressing the desirable activity.

The skilled artisan will recognize that the relevant assay, screening or selection method will vary depending upon the desired host organism and other parameters known in the art. It is normally advantageous to employ an assay that can be practiced in a high-throughput format.

In high-throughput assays, it is possible to screen up to several thousand different variants in a single day. For example, each well of a microtiter plate can be used to run a separate assay, or, if concentration or incubation time effects are to be observed, every 5-10 wells can test a single variant.

In addition to fluidic approaches, it is possible, as mentioned above, simply to grow cells on media plates that select for the desired enzymatic or metabolic function. This approach offers a simple and high-throughput screening method.

A number of robotic systems have also been developed for solution phase chemistries useful in assay systems. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; and Orca, Hewlett-Packard, Palo Alto, Calif.) which mimic the manual synthetic operations performed by a scientist. Any of the above devices are suitable for application to the present invention. The nature and implementation of modifications to these devices (if any) so that they can operate as discussed herein with reference to the integrated system will be apparent to persons skilled in the relevant art.

High-throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the particular assay. These configurable systems provide high throughput and rapid start up as well as a high degree of flexibility and customization.

The manufacturers of such systems provide detailed protocols for the various high throughput devices. Thus, for example, Zymark Corp. provides technical bulletins describing screening systems for detecting the modulation of gene transcription, ligand binding, and the like. Microfluidic approaches to reagent manipulation have also been developed, e.g., by Caliper Technologies (Mountain View, Calif.).

Optical images viewed (and, optionally, recorded) by a camera or other recording device (e.g., a photodiode and data storage device) are optionally further processed in any of the embodiments herein, e.g., by digitizing the image and/or storing and analyzing the image on a computer. A variety of commercially available peripheral equipment and software is available for digitizing, storing and analyzing a digitized video or digitized optical image, e.g., using PC (Intel x86 or Pentium chip compatible DOS™, OS™ WINDOWS™, WINDOWS NT™ or WINDOWS 95™ based machines), MACINTOSH™, or UNIX based (e.g., SUN™ work station) computers.

One conventional system carries light from the assay device to a cooled charge-coupled device (CCD) camera, a common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the invention are easily used for viewing any sample, e.g. by fluorescent or dark field microscopic techniques.

b. Sequence Identity

An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein or biologically active portion thereof is recombinantly produced, optimally culture medium represents less than about 30%, 20%, 10%, 5%, or 1% (by dry weight) of chemical precursors or non-protein-of-interest chemicals.

Fragments and variants of the disclosed amino acid-N-acetyltransferase polynucleotides and proteins encoded thereby are also encompassed. By “fragment” is intended a portion of the polynucleotide or a portion of the amino acid sequence and hence protein encoded thereby. Fragments of a polynucleotide may encode protein fragments that retain amino acid-N-acetyltransferase activity.

A fragment of an amino acid-N-acetyltransferase polynucleotide that encodes a biologically active portion of an amino acid-N-acetyltransferase protein will encode at least 15, 25, 30, 50, 100, 150, 200, or 250 contiguous amino acids, or up to the total number of amino acids present in a full-length amino acid-N-acetyltransferase protein. Fragments of an amino acid-N-acetyltransferase polynucleotide that are useful as hybridization probes or PCR primers generally need not encode a biologically active portion of an amino acid-α-N-acetyltransferase.

Thus, a fragment of an amino acid-N-acetyltransferase polynucleotide may encode a biologically active portion of an amino acid-N-acetyltransferase, or it may be a fragment that can be used as a hybridization probe or PCR primer using methods disclosed below. A biologically active portion of an amino acid-N-acetyltransferase can be prepared by isolating a portion of one of the amino acid-N-acetyltransferase polynucleotide, expressing the encoded portion of the amino acid-N-acetyltransferase protein (e.g., by recombinant expression in vitro), and assessing the activity of the encoded portion of the amino acid-N-acetyltransferase. Polynucleotides that are fragments of an amino acid-N-acetyltransferase nucleotide sequence comprise at least 16, 20, 50, 75, 100, 150, 200, 250, 300, 350, 400, 450, 500, 550, 600, 650, 700, 800, 900, 1,000, 1,100, 1,200, 1,300, or 1,400 contiguous nucleotides, or up to the number of nucleotides present in a full-length amino acid-α-N-acetyltransferase polynucleotide disclosed herein.

“Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the amino acid-α-N-acetyltransferase polypeptides. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotide, such as those generated, for example, by using site-directed mutagenesis but which still encode amino acid-N-acetyltransferase protein. Generally, variants of a particular polynucleotide will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, an isolated polynucleotide that encodes a polypeptide with a given percent sequence identity an amino acid-N-acetyltransferase polypeptide. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the reference protein by deletion or addition of one or more amino acids at one or more internal sites in the reference protein and/or substitution of one or more amino acids at one or more sites in the reference protein. Variant proteins encompassed by the present invention are biologically active, that is they continue to possess the desired biological activity of the reference protein, that is, amino acid-N-acetyltransferase activity as described herein. Such variants may result from, for example, genetic polymorphism or from human manipulation. Biologically active variants of an amino acid-N-acetyltransferase protein of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein may differ from that protein by as few as 1-15 amino acid residues, as few as 1-10, such as 6-10, as few as 5, as few as 4, 3, 2, or even 1 amino acid residue.

The proteins may be altered in various ways including amino acid substitutions, deletions, truncations, and insertions. Methods for such manipulations are generally known in the art. For example, amino acid sequence variants and fragments of the amino acid-α-N-acetyltransferase proteins can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Thus, the genes and polynucleotides of the invention include both the naturally occurring sequences as well as mutant forms. Likewise, the proteins of the invention encompass both naturally occurring proteins as well as variations and modified forms thereof. Such variants will continue to possess the desired amino acid-N-acetyltransferase activity. Obviously, the mutations that will be made in the DNA encoding the variant must not place the sequence out of reading frame and optimally will not create complementary regions that could produce secondary mRNA structure. See, EP Patent Application Publication No. 75,444.

The deletions, insertions, and substitutions of the protein sequence encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. Assays for measuring the acetylation of the α-amine of free amino acids or the ε-amine of free lysine are disclosed elsewhere herein.

Variant polynucleotides and proteins also encompass sequences and proteins derived from a mutagenic and recombinogenic procedure such as DNA shuffling. With such a procedure, one or more different amino acid-N-acetyltransferase coding sequences can be manipulated to create a new amino acid-N-acetyltransferase possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between a first amino acid-N-acetyltransferase gene and other known amino acid-N-acetyltransferase genes to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The following terms are used to describe the sequence relationships between two or more polynucleotides or polypeptides: (a) “reference sequence”, (b) “comparison window”, (c) “sequence identity”, and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al. (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

### III. Plants, Plant Parts, Seeds, and Grain

Plants, plant cells, plant parts, seed, grain, and plant material having increased levels of at least one selected free ε- and/or α-N-acetylated amino acid are provided. Further provided are products such as, but not limited to, food or feed products (fresh or processed) comprising or derived from plant material. In specific embodiments, the plants, plant parts, seed and/or grain have a total free α-N-acetylated amino acid content of at least about 100 ppm to about 10,000 ppm or at least 200 ppm. In further embodiments, the transgenic plant, plant cell, plant part, grain or seed comprises at least about 100 ppm to 1000 ppm of free α-N-acetyl-methionine; at least about 100 ppm to about 4000 of free α-N-acetyl-lysine, at least about 100 ppm to about 3000 ppm of free α-N-acetyl-threonine; at least about 100 ppm to about 600 ppm of free α-N-acetyl-tryptophan, at least about 100 ppm to about 1000 ppm of free α-N-acetyl-cysteine, or any combination thereof.

In other embodiments, the plant, plant part, seed or grain have a ratio of free α-N-acetylated amino acid content to free non-α-N-acetylated amino acid content of about 3:1 to about 1000:1; at least about 1:1 to about 1000:1 or greater; about 1:1, about 0.5:1 to about 100:1, about 0.5:1 to about 20:1, about 1:1 to about 500:1, about 500:1 to about 800:1, about 800:1 to about 1000:1, or about 900:1 to about 1100. In further embodiments, the plant, plant cell, seed or grain comprises a free α-N-acetyl-methionine to free non-α-N-acetylated methionine ratio of at least about 100:1 to about 1000:1 or greater, about 20:1, about 10:1 to about 1000:1, about 10:1 to about 50:1, about 50:1 to about 500:1, or about 500:1 to about 1000:1; a free α-N-acetyl-lysine to free non-α-N-acetylated lysine ratio of at least about 20:1 to about 200:1 or greater; at least about 185:1, about 150:1 to about 190:1 or at least about 75:1 to about 125:1; a free α-N-acetyl-threonine to free non-α-N-acetylated threonine ratio of at least about 10:1 to about 120:1 or greater; about 124:1, about 10:1 to about 50:1, about 50:1 to about 100:1, about 100:1 to about 125:1, about 120:1 to about 130:1; a free α-N-acetyl-tryptophan to free non-α-N-acetylated tryptophan ratio of at least about 1.2:1 to about 3:1 or greater, about 2.61:1 or about 2:1 to about 2.5:1, or about 1.7 to about 2.8; a free α-N-acetyl-cysteine to free non-α-N-acetylated cysteine ratio of at least about 100:1 to about 1000:1 or greater, about 1049:1, about 100:1 to about 500:1, about 500:1 to about 1000:1, about 900:1 to about 1050:1, about 1025:1 to about 1055:1; and/or any combination thereof.

In one embodiment, the ratio comprises at least about 1000:1.

While any means can be used to produce the transgenic seed or grain having the increased level of at least one free ε- and/or α-N-acetylated amino acid, in one embodiment, the transgenic plant, plant part, seed or grain has stably incorporated into its genome a heterologous polynucleotide encoding a polypeptide encoding an amino acid-N-acetyltransferase polypeptide (i.e., such as an amino acid-α-N-acetyltransferase) operably linked to a promoter active in the seed of the plant. Various amino acid-N-acetyltransferase polypeptides that can be used are disclosed elsewhere herein. In specific embodiments, the plant, plant part, seed or grain having the heterologous polynucleotide are characterized as having an increased level of free α-N-acetyl-methionine or free α-N-acetyl-lysine when compared to a control plant not expressing the heterologous polynucleotide. In other embodiments, the plant, plant part, seed or gain having the heterologous polynucleotide are characterized as having a free α-N-acetylated amino acid content of at least about 100 ppm to about 1000 ppm and/or a ratio of free α-N-acetylated amino acid content to free non-α-N-acetylated amino acid content of about 3 to about 1000.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, seed coat, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced or heterologous polynucleotides disclosed herein.

Any plant species can be transformed, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include plants that produce cereal grains (i.e., barley, maize, millet, oats, rye, rice sorghum, triticale, and wheat), oil-seed plants (i.e., canola, cotton, linseed, rapeseed, safflower, soybean, sunflower, Brassica, maize, alfalfa, palm, coconut,), and pulses (i.e., leguminous plants, such as, beans and peas). Beans include guar, locust bean, fenugreek, soybean, lupins, peanuts, garden beans, cowpea, mungbean, lima bean, fava bean, lentils, chickpea, etc.)

A “subject plant or plant cell” is one in which an alteration, such as transformation or introduction of a polypeptide, has occurred, or is a plant or plant cell which is descended from a plant or cell so altered and which comprises the alteration. A “control” or “control plant” or “control plant cell” provides a reference point for measuring changes in phenotype of the subject plant or plant cell.

A control plant or plant cell may comprise, for example: (a) a wild-type plant or cell, i.e., of the same genotype as the starting material for the alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

### IV. Polynucleotide Constructs

The use of the term “polynucleotide” is not intended to be limited to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The polynucleotides employed can be provided in expression cassettes for expression in the plant of interest. The cassette will include 5′ and 3′ regulatory sequences operably linked to a polynucleotide of the invention. “Operably linked” is intended to mean a functional linkage between two or more elements. For example, an operable linkage between a polynucleotide of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the polynucleotide to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a polynucleotide of the invention, and a transcriptional and translational termination region (i.e., termination region) functional in plants. The regulatory regions (i.e., promoters, transcriptional regulatory regions, and translational termination regions) and/or the polynucleotide of the invention may be native/analogous to the host cell or to each other. Alternatively, the regulatory regions and/or the polynucleotide of the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

While it may be optimal to express the sequences using heterologous promoters, the native promoter sequences may be used. Such constructs can change expression levels of the protein in the plant or plant cell. Thus, the phenotype of the plant or plant cell can be altered.

The termination region may be native with the transcriptional initiation region, may be native with the operably linked polynucleotide of interest, may be native with the plant host, or may be derived from another source (i.e., foreign or heterologous) to the promoter, the polynucleotide of interest, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention, including the native promoter of the polynucleotide sequence of interest. The promoters can be selected based on the desired outcome. The nucleic acids can be combined with constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced expression of a polynucleotide within a particular plant tissue. “Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108, herein incorporated by reference. Such seed-preferred promoters include, but are not limited to, Cim1 (cytokinin-induced message); cZ19B1 (maize 19 kDa zein); milps (myo-inositol-1-phosphate synthase) (see WO 00/11177 and U.S. Pat. No. 6,225,529; herein incorporated by reference). Gamma-zein is an endosperm-specific promoter. Globulin 1 (Glb-1) is a representative embryo-specific promoter. For dicots, seed-specific promoters include, but are not limited to, bean β-phaseolin, napin, β-conglycinin, soybean lectin, cruciferin, annexin, glycinin, P34, Kunitz trypsin inhibitor 3 and the like. For monocots, seed-specific promoters include, but are not limited to, maize 15 kDa zein, 22 kDa zein, 27 kDa zein, gamma-zein, waxy, shrunken 1, shrunken 2, Globulin 1, etc. See also WO 00/12733, where seed-preferred promoters from end1 and end2 genes are disclosed; herein incorporated by reference. The oleosin promoter and the Lpt2 promoters (for example, U.S. Pat. No. 6,013,862, WO95/15389 and WO 95/23230) can also be used.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference. The above list of selectable marker genes is not meant to be limiting. Any selectable marker gene can be used in the present invention.

In certain embodiments, the polynucleotides employed in the invention can be stacked to create plants with a desired trait. A trait, as used herein, refers to the phenotype derived from a particular sequence or groups of sequences. For example, polynucleotide encoding an amino acid acetyltransferase polypeptide may be stacked with one or more additional polynucleotides of interest. These stacked combinations can be created by any method including, but not limited to, cross-breeding plants by any conventional methodology, or genetic transformation. If the sequences are stacked by genetically transforming the plants, the polynucleotide sequences can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference.

### V. Methods of Introducing

Methods of the invention increase the level of an amino acid-α-N-acetyltransferase polypeptide. Such methods can be achieved by introducing a polypeptide or polynucleotide into a plant. “Introducing” is intended to mean presenting to the plant the polynucleotide or polypeptide in such a manner that the sequence gains access to the interior of a cell of the plant. The methods do not depend on a particular method for introducing a sequence into a plant, only that the polynucleotide or polypeptide gains access to the interior of at least one cell of the plant. Methods for introducing a polynucleotide or a polypeptide into a plant are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski et al. (1984) EMBO J. 3:2717-2722), and ballistic particle acceleration (see, for example, U.S. Pat. Nos. 4,945,050; U.S. Pat. No. 5,879,918; U.S. Pat. No. 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet.

22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In specific embodiments, the sequences employed in the invention can be provided to a plant using a variety of transient transformation methods. Such transient transformation methods include, but are not limited to, the introduction of a protein or variants and fragments thereof directly into the plant or the introduction of the a transcript encoding the protein into the plant. Such methods include, for example, microinjection or particle bombardment. See, for example, Crossway et al. (1986) Mol. Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference. Alternatively, a polynucleotide can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which its released to become integrated into the genome is greatly reduced. Such methods include the use particles coated with polyethylimine (PEI; Sigma #P3143).

In other embodiments, the polynucleotide of the invention may be introduced into plants by contacting plants with a virus or viral nucleic acids. Generally, such methods involve incorporating a nucleotide construct of the invention within a viral DNA or RNA molecule. It is recognized that the polypeptides employed in the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide of the invention can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide of the invention, for example, an expression cassette of the invention, stably incorporated into their genome.

### V. Methods of Use

a. Methods for Increasing the Level of an Amino Acid-N-Acetyltransferase in a Plant or Plant Part

A method for increasing the level of a polypeptide comprising an amino acid-N-acetyltransferase (such as an amino acid-α-N-acetyltransferase) or a functional variant or fragment thereof in a plant is provided.

An “increased level” or “increasing the level” of a polypeptide refers to any increase in the expression, concentration, or activity of a gene product, including any relative increment in expression, concentration or activity. Any method or composition that increases expression of a target gene product, either at the level of transcription or translation, or increases the activity of the target gene product can be used to achieve increased expression, concentration, activity of the target gene product. In general, the level is increased by at least 1%, 5%, 10%, 20%, 30%, 40%, 50%, 60%, 70%, 80%, 90% or greater relative to a native control plant, plant part, seed, grain, or cell.

The level of the polypeptide encoding an amino acid-N-acetyltransferase (such as an amino acid-α-N-acetyltransferase) may be measured directly, for example, by assaying for the concentration of the polypeptide in the plant, or indirectly, for example, by measuring the amount of activity of the polypeptide in the plant. Methods for determining the activity of these polypeptides are described elsewhere herein.

In specific embodiments, the polynucleotide encoding the amino acid-N-acetyltransferase (such as an amino acid-α-N-acetyltransferase) is introduced into the plant cell. Subsequently, a plant cell having the introduced amino acid-α-N-acetyltransferase is selected using methods known to those of skill in the art such as, but not limited to, Southern blot analysis, DNA sequencing, PCR analysis, or phenotypic analysis. A plant or plant part altered or modified by the foregoing embodiments is grown under plant forming conditions for a time sufficient to increase the level of the targeted polypeptide in the plant. Plant forming conditions are well known in the art and discussed briefly elsewhere herein.

It is therefore recognized that methods of the present invention do not depend on the incorporation of the entire polynucleotide into the genome, only that the plant or cell thereof is altered as a result of the introduction of a polynucleotide into a cell. In one embodiment of the invention, the genome may be altered following the introduction of the polynucleotide into a cell. For example, the polynucleotide, or any part thereof, may incorporate into the genome of the plant. Alterations to the genome of the present invention include, but are not limited to, additions, deletions, and substitutions of nucleotides into the genome. While the methods of the present invention do not depend on additions, deletions, and substitutions of any particular number of nucleotides, it is recognized that such additions, deletions, or substitutions comprises at least one nucleotide.

As discussed elsewhere herein, many methods are known in the art for providing a polypeptide to a plant including, but not limited to, direct introduction of the polypeptide into the plant, introducing into the plant (transiently or stably) a polynucleotide construct encoding a polypeptide having the appropriate activity as described elsewhere herein. It is also recognized that the methods of the invention may employ a polynucleotide that is not capable of directing, in the transformed plant, the expression of a protein or an RNA. Thus, the level of an amino acid acetyltransferase may be increased by altering the gene encoding the respective polypeptide or its promoter. See, e.g., Kmiec, U.S. Pat. No. 5,565,350; Zarling et al., PCT/US93/03868. Therefore, mutagenized plants that carry mutations in a polynucleotide encoding an amino acid acetyltransferase where the mutations increase expression of the amino acid acetyltransferase are provided.

b. Methods to Improve Grain and/or Feed Quality

Further provided are methods to improve the nutrient availability of seed or grain by increasing the content of one or more selected free ε- and/or α-N-acetylated amino acids in the plant, plant part, or seed of the plant. The plant, plant part, seed and/or grain having the increased content of one or more selected free ε- and/or α-N-acetylated amino acids finds use as a food source, animal feed and or as a feed supplement with higher nutritional value. Accordingly, methods are provided to improve the tissue quality or nutritional health of an animal by feeding an animal a diet having an elevated level of one or more selected free ε- and/or α-N-acetylated amino acid. Such methods comprise feeding the animal a diet comprising a sufficient amount of a grain which comprises an elevated level of one or more selected free and/ε- or α-N-acetylated amino acid(s). In specific embodiments, the feed comprises grain having an increased level of an amino acid-N-acetyltransferase polypeptide (such as an amino acid-α-N-acetyltransferase polypeptide).

In specific embodiments, the food source, animal feed, or supplement comprises grain from wheat or rice having at least an increased level of free α-N-acetyl-lysine. In other embodiments, the food source, animal feed or supplement comprises grain from maize having at least an increased level of free α-N-acetyl-lysine and/or free α-N-acetyl-tryptophane. In other embodiments, the food source, animal feed or supplement comprises grain from soybean having at least an increased level of at least free α-N-acetyl-cysteine, free α-N-acetyl-methionine, free α-N-acetyl-lysine, and/or free α-N-acetyl-tryptophan. In other embodiments, the food source, animal feed or supplement comprises grain from legumes having an increased level of free α-N-acetyl-methionine and/or free α-N-acetyl-cysteine.

In one embodiment, such methods comprise feeding a diet comprising a sufficient amount of a grain where the grain comprises a polynucleotide encoding an amino acid-α-N-acetyltransferase or a grain having an increased level of a selected free α-N-acetylated amino acid. The feed employed in the diet can comprise about 5%, 10%, 20%, 30%, 40%, 50%, 60%, 70%, 80%, 90%, or 100% of the grain having the increased level of the desired free α-N-acetylated amino acid. In other embodiments, the feed employed in the diet can comprise about 1 to about 15%, about 10 to about 25%, about 20 to about 35%, about 30 to about 45%, about 40% to about 55%, about 50 to about 65%, about 60 to about 75%, about 70 to about 85%, about 80% to about 95% or about 90% to 100% of the grain with the increased level of the desired free α-N-acetylated amino acid.

The diet can be supplied for any number of days. Accordingly, in specific embodiments, the diet if feed for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 weeks or longer.

Animals of interest include, but are not limited to, humans, ruminant animals, including, but not limited to, cattle, bison, or lamb, as well as, non-ruminant animals including, but not limited to, swine, poultry (i.e., chickens, layer hens, turkey, ostriches and emu) or fish.

Embodiments of the present invention are further defined in the following Examples. It should be understood that these Examples are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### EXPERIMENTAL

### Example 1

### Transformation and Regeneration of Maize

Immature maize embryos from greenhouse donor plants are bombarded with a plasmid containing an amino acid-N-acetyltransferase operably linked to a promoter of interest and the selectable marker gene PAT (Wohlleben et al. (1988) Gene 70:25-37), which confers resistance to the herbicide Bialaphos. Alternatively, the selectable marker gene is provided on a separate plasmid. Transformation is performed as follows. Media recipes follow below.

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5 cm target zone in preparation for bombardment.

A plasmid vector comprising the amino acid-N-acetyltransferase operably linked to the promoter of interest is made. This plasmid DNA plus plasmid DNA containing a PAT selectable marker is precipitated onto 1.1 μm (average diameter) tungsten pellets using a CaCl₂ precipitation procedure as follows: 100 μl prepared tungsten particles in water; 10 μl (1 μg) DNA in Tris EDTA buffer (1 μg total DNA); 100 μl 2.5 M CaCl₂; and, 10 μl 0.1 M spermidine.

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macro carrier and allowed to dry about 2 minutes before bombardment.

The sample plates are bombarded at level #4 in a particle gun. All samples receive a single shot at 650 PSI, with a total of ten aliquots taken from each tube of prepared particles/DNA.

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for an increased level in the desired acetylated amino acid(s).

Bombardment medium (560Y) comprises 4.0 g/l N6 basal salts (SIGMA C-1416), 1.0 ml/l Eriksson's Vitamin Mix (1000× SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

Bombardment medium (560Y) comprises 4.0 g/l N6 basal salts (SIGMA C-1416), 1.0 ml/l Eriksson's Vitamin Mix (1000× SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/1 myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

TABLE 1
Non-limiting representative levels of
amino acid content in soybean grain.

| | Content, Mg/gDW | | | | | |
|---|---|---|---|---|---|---|
| | Free | | N-acetyl | New | % | Acetyl/free |
| Amino acid | (1) | Total | (2) | total | increase | Non-acetyl |
| cysteine | 0.001 | 7 | 1.05 | 8.05 | 15 | 1049 |
| methionine | 0.05 | 7 | 1.00 | 8.05 | 15 | 20 |
| lysine | 0.025 | 31 | 4.63 | 35.7 | 15 | 185 |
| threonine | 0.022 | 18.4 | 2.74 | 21.2 | 15 | 124 |
| tryptophan | 0.21 | 5.05 | 0.55 | 5.81 | 15 | 2.61 |
| aspartate | 0.295 | 52.1 | 0.17 | 52.27 | 0.326 | 0.58 |
| glutamate | 0.4 | 81.6 | 0.007 | 81.607 | 0.0086 | 0.02 |

(1) Takahashi et al. (2003) Planta 217: 577-586
(2) amount calculated to results in a 15% increase in total

### Example 2

### Agrobacterium-Mediated Transformation of Maize

For Agrobacterium-mediated transformation of maize with an amino acid-N-acetyltransferase, the method of Zhao is employed (U.S. Pat. No. 5,981,840, and PCT patent publication WO98/32326; the contents of which are hereby incorporated by reference). Briefly, immature embryos are isolated from maize and the embryos contacted with a suspension of Agrobacterium, where the bacteria are capable of transferring the amino acid-N-acetyltransferase to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). The immature embryos are cultured on solid medium with a selective agent resulting in the selective growth of transformed cells. The callus is then regenerated into plants (step 5: the regeneration step), and calli grown on selective medium are cultured on solid medium to regenerate the plants.

### Example 3

### Soybean Embryo Transformation

Soybean embryogenic suspension cultures (cv. Jack) are maintained in 35 ml liquid medium SB196 (see recipes below) on rotary shaker, 150 rpm, 26° C. with cool white fluorescent lights on 16:8 hr day/night photoperiod at light intensity of 60-85 μE/m2/s. Cultures are subcultured every 7 days to two weeks by inoculating approximately 35 mg of tissue into 35 ml of fresh liquid SB196 (the preferred subculture interval is every 7 days). Soybean embryogenic suspension cultures are transformed with the plasmids and DNA fragments described in the following examples by the method of particle gun bombardment (Klein et al. (1987) Nature, 327:70). Soybean cultures are initiated twice each month with 5-7 days between each initiation.

Pods with immature seeds from available soybean plants 45-55 days after planting are picked, removed from their shells and placed into a sterilized magenta box. The soybean seeds are sterilized by shaking them for 15 minutes in a 5% Clorox solution with 1 drop of ivory soap (95 ml of autoclaved distilled water plus 5 ml Clorox and 1 drop of soap). Mix well. Seeds are rinsed using 2 l-liter bottles of sterile distilled water and those less than 4 mm are placed on individual microscope slides. The small end of the seed are cut and the cotyledons pressed out of the seed coat. Cotyledons are transferred to plates containing SB1 medium (25-30 cotyledons per plate). Plates are wrapped with fiber tape and stored for 8 weeks. After this time secondary embryos are cut and placed into SB196 liquid media for 7 days.

Either an intact plasmid or a DNA plasmid fragment containing the genes of interest and the selectable marker gene are used for bombardment. Plasmid DNA for bombardment are routinely prepared and purified using the method described in the Promega™ Protocols and Applications Guide, Second Edition (page 106). Fragments of the plasmids carrying the amino acid-α-N-acetyltransferase are obtained by gel isolation of double digested plasmids.

In each case, 100 ug of plasmid DNA is digested in 0.5 ml of the specific enzyme mix that is appropriate for the plasmid of interest. The resulting DNA fragments are separated by gel electrophoresis on 1% SeaPlaque GTG agarose (BioWhitaker Molecular Applications) and the DNA fragments containing the amino acid-N-acetyltransferase are cut from the agarose gel. DNA is purified from the agarose using the GELase digesting enzyme following the manufacturer's protocol.

A 50 μl aliquot of sterile distilled water containing 3 mg of gold particles (3 mg gold) is added to 5 μl of a 1 μg/μl DNA solution (either intact plasmid or DNA fragment prepared as described above), 50 μl 2.5M CaCl₂ and 20 μl of 0.1 M spermidine. The mixture is shaken 3 min on level 3 of a vortex shaker and spun for 10 sec in a bench microfuge. After a wash with 400 μl 100% ethanol the pellet is suspended by sonication in 40 μl of 100% ethanol. Five μl of DNA suspension is dispensed to each flying disk of the Biolistic PDS1000/HE instrument disk. Each 5 μl aliquot contains approximately 0.375 mg gold per bombardment (i.e. per disk).

Approximately 150-200 mg of 7 day old embryonic suspension cultures are placed in an empty, sterile 60×15 mm petri dish and the dish covered with plastic mesh. Tissue is bombarded 1 or 2 shots per plate with membrane rupture pressure set at 1100 PSI and the chamber evacuated to a vacuum of 27-28 inches of mercury. Tissue is placed approximately 3.5 inches from the retaining/stopping screen.

Transformed embryos were selected either using hygromycin (when the hygromycin phosphotransferase, HPT, gene was used as the selectable marker) or chlorsulfuron (when the acetolactate synthase, ALS, gene was used as the selectable marker).

Following bombardment, the tissue is placed into fresh SB196 media and cultured as described above. Six days post-bombardment, the SB196 is exchanged with fresh SB196 containing a selection agent of 30 mg/L hygromycin. The selection media is refreshed weekly. Four to six weeks post selection, green, transformed tissue may be observed growing from untransformed, necrotic embryogenic clusters. Isolated, green tissue is removed and inoculated into multiwell plates to generate new, clonally propagated, transformed embryogenic suspension cultures.

Following bombardment, the tissue is divided between 2 flasks with fresh SB196 media and cultured as described above. Six to seven days post-bombardment, the SB196 is exchanged with fresh SB196 containing selection agent of 100 ng/ml Chlorsulfuron. The selection media is refreshed weekly. Four to six weeks post selection, green, transformed tissue may be observed growing from untransformed, necrotic embryogenic clusters. Isolated, green tissue is removed and inoculated into multiwell plates containing SB196 to generate new, clonally propagated, transformed embryogenic suspension cultures.

In order to obtain whole plants from embryogenic suspension cultures, the tissue must be regenerated.

Embryos are cultured for 4-6 weeks at 26° C. in SB196 under cool white fluorescent (Phillips cool white Econowatt F40/CW/RS/EW) and Agro (Phillips F40 Agro) bulbs (40 watt) on a 16:8 hr photoperiod with light intensity of 90-120 uE/m2s. After this time embryo clusters are removed to a solid agar media, SB166, for 1-2 weeks. Clusters are then subcultured to medium SB103 for 3 weeks. During this period, individual embryos can be removed from the clusters and screened for an increased acetylated amino acid(s) levels. It should be noted that any detectable phenotype, resulting from the expression of the genes of interest, could be screened at this stage.

Matured individual embryos are desiccated by placing them into an empty, small petri dish (35×10 mm) for approximately 4-7 days. The plates are sealed with fiber tape (creating a small humidity chamber). Desiccated embryos are planted into SB71-4 medium where they were left to germinate under the same culture conditions described above. Germinated plantlets are removed from germination medium and rinsed thoroughly with water and then planted in Redi-Earth in 24-cell pack tray, covered with clear plastic dome. After 2 weeks the dome is removed and plants hardened off for a further week. If plantlets looked hardy they are transplanted to 10″ pot of Redi-Earth with up to 3 plantlets per pot. After 10 to 16 weeks, mature seeds are harvested, chipped and analyzed for proteins.

SB 196 - FN Lite liquid proliferation medium (per liter) -

| | | |
|---|---|---|
| MS FeEDTA - 100x Stock 1 | 10 | ml |
| MS Sulfate - 100x Stock 2 | 10 | ml |
| FN Lite Halides - 100x Stock 3 | 10 | ml |
| FN Lite P, B, Mo - 100x Stock 4 | 10 | ml |
| B5 vitamins (1 ml/L) | 1.0 | ml |
| 2,4-D (10 mg/L final concentration) | 1.0 | ml |
| KNO3 | 2.83 | gm |
| (NH4)2 SO 4 | 0.463 | gm |
| Asparagine | 1.0 | gm |
| Sucrose (1%) | 10 | gm |
| pH 5.8 | | |

### FN Lite Stock Solutions

| Stock # | | 1000 ml | | 500 ml | |
|---|---|---|---|---|---|
| 1 | MS Fe EDTA 100x Stock | | | | |
| | Na₂ EDTA* | 3.724 | g | 1.862 | g |
| | FeSO₄—7H₂O | 2.784 | g | 1.392 | g |
| 2 | MS Sulfate 100x stock | | | | |
| | MgSO₄—7H₂O | 37.0 | g | 18.5 | g |
| | MnSO₄—H₂O | 1.69 | g | 0.845 | g |
| | ZnSO₄—7H₂O | 0.86 | g | 0.43 | g |
| | CuSO₄—5H₂O | 0.0025 | g | 0.00125 | g |
| 3 | FN Lite Halides 100x Stock | | | | |
| | CaCl₂—2H₂O | 30.0 | g | 15.0 | g |
| | KI | 0.083 | g | 0.0715 | g |
| | CoCl₂—6H₂O | 0.0025 | g | 0.00125 | g |
| 4 | FN Lite P, B, Mo 100x Stock | | | | |
| | KH₂PO₄ | 18.5 | g | 9.25 | g |
| | H₃BO₃ | 0.62 | g | 0.31 | g |
| | Na₂MoO₄—2H₂O | 0.025 | g | 0.0125 | g |

*Add first, dissolve in dark bottle while stirring

SB1 solid medium (per liter) comprises: 1 pkg. MS salts (Gibco/BRL—Cat# 11117-066); 1 ml B5 vitamins 1000× stock; 31.5 g sucrose; 2 ml 2,4-D (20 mg/L final concentration); pH 5.7; and, 8 g TC agar.

SB 166 solid medium (per liter) comprises: 1 pkg. MS salts (Gibco/BRL—Cat# 11117-066); 1 ml B5 vitamins 1000× stock; 60 g maltose; 750 mg MgCl2 hexahydrate; 5 g activated charcoal; pH 5.7; and, 2 g gelrite.

SB 103 solid medium (per liter) comprises: 1 pkg. MS salts (Gibco/BRL—Cat# 11117-066); 1 ml B5 vitamins 1000× stock; 60 g maltose; 750 mg MgCl2 hexahydrate; pH 5.7; and, 2 g gelrite.

SB 71-4 solid medium (per liter) comprises: 1 bottle Gamborg's B5 salts w/ sucrose (Gibco/BRL—Cat# 21153-036); pH 5.7; and, 5 g TC agar.

2,4-D stock is obtained premade from Phytotech cat# D 295—concentration is 1 mg/ml.

B5 Vitamins Stock (per 100 ml) which is stored in aliquots at −20 C comprises: 10 g myo-inositol; 100 mg nicotinic acid; 100 mg pyridoxine HCl; and, 1 g thiamine. If the solution does not dissolve quickly enough, apply a low level of heat via the hot stir plate. Chlorsulfuron Stock comprises 1 mg/ml in 0.01 N Ammonium Hydroxide

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A transgenic seed or transgenic grain having a free α-N-acetylated amino acid content of at least 220 ppm or a transgenic seed or transgenic grain having a ratio of free α-N-acetylated amino acid content to free non-α-N-acetylated amino acid content of about 1:1.

2. The transgenic seed or transgenic grain of claim 1, wherein said free α-N-acetylated amino acid content comprises
a) at least 100 ppm of free α-N-acetyl-methionine;
b) at least 100 ppm of free α-N-acetyl-lysine;
c) at least 1000 ppm of free α-N-acetyl-threonine;
d) at least 100 ppm of free α-N-acetyl-tryptophan; or,
e) at least 100 ppm of free α-N-acetyl-cysteine.

3. The transgenic seed or transgenic grain of claim 1, wherein said free α-N-acetylated amino acid content is selected from the group consisting of:
a) a free α-N-acetyl-methionine to free non-α-N-acetylated methionine ratio of at least about 100:1;
b) a free α-N-acetyl-lysine to free non-α-N-acetylated lysine ratio of at least about 20:1;
c) a free α-N-acetyl-threonine to free non-α-N-acetylated threonine ratio of at least about 10:1;
d) a free α-N-acetyl-tryptophan to free non-α-N-acetylated tryptophan ratio of at least about 1.2:1; or
e) a free α-N-acetyl-cysteine to free non-α-N-acetylated cysteine ratio of at least about 100:1.

4. A plant having stably incorporated into its genome a heterologous polynucleotide encoding an amino acid-α-N-acetyltransferase polypeptide operably linked to a promoter active in the seed of said plant, wherein the seed of said plant comprises
a) an increased level of free α-N-acetyl-methionine or free α-N-acetyl-lysine when compared to a control plant not expressing said heterologous polynucleotide;
b) a free α-N-acetylated amino acid content of at least 220 ppm; or,
c) a ratio of free α-N-acetylated amino acid content to free non-α-N-acetylated amino acid content of about 1:1.

5. The plant of claim 4, wherein said promoter comprises a seed-preferred promoter.

6. The plant of claim 4, wherein the seed of said plant comprises
a) a free α-N-acetyl-methionine to free non-α-N-acetylated methionine ratio of at least about 20:1;
b) a free α-N-acetyl-lysine to free non-α-N-acetylated lysine ratio of at least about 20:1;
c) a free α-N-acetyl-threonine to free non-α-N-acetylated threonine ratio of at least about 10:1
d) a free α-N-acetyl-tryptophan to free non-α-N-acetylated tryptophan ratio of at least about 1.2:1
e) a free α-N-acetyl-cysteine to free non-α-N-acetylated cysteine ratio of at least about 100:1.

7. The plant of claim 4, wherein said free α-N-acetylated amino acid content comprises
a) at least 100 ppm of free α-N-acetyl-methionine;
b) at least 100 ppm of free α-N-acetyl-lysine;
c) at least 1000 ppm of free α-N-acetyl-threonine;
d) at least 100 ppm of free α-N-acetyl-tryptophan; or,
e) at least 100 ppm of free α-N-acetyl-cysteine.

8. A transgenic seed or transgenic grain produced by the plant of claim 4 having stably incorporated into its genome said polynucleotide.

9. A plant having stably incorporated into its genome a first heterologous polynucleotide encoding a first amino acid-α-N-acetyltransferase polypeptide operably linked to a first promoter active in the seed of said plant and a second heterologous polynucleotide encoding a second amino acid-α-N-acetyltransferase polypeptide operably linked to a second promoter active in the seed of said plant, wherein said first and said second amino acid-α-N-acetyltransferase polypeptide acetylate the α-amine of distinct amino acids.

10. The plant of claim 9, wherein at least one of said first or said second amino acid-α-N-acetyltransferase polypeptides acetylates the α-amino of at least one of methionine, lysine, threonine, cysteine, or, tryptophan.

11. The plant of claim 9, wherein said first or said second promoter is a seed-preferred promoter.

12. The plant of claim 9, wherein the transgenic seed or the transgenic grain of said plant comprises a free α-N-acetylated amino acid content of at least 220 ppm.

13. The plant of claim 12, wherein the transgenic seed or the transgenic grain of said plant comprises a free α-N-acetylated amino acid content of
a) at least about 100 ppm of free α-N-acetyl-methionine;
b) at least about 100 ppm of free α-N-acetyl-lysine;
c) at least about 1000 ppm of free α-N-acetyl-threonine;
d) at least about 100 ppm of free α-N-acetyl-tryptophan; or,
e) at least about 100 ppm of free α-N-acetyl-cysteine.

14. The plant of claim 9, wherein the transgenic seed or the transgenic grain of said plant comprises a free α-N-acetylated amino acid content to a free non-α-N-acetylated amino acid content of about 1:1.

15. The plant of claim 14, wherein said transgenic seed or said transgenic grain comprises
a) a free α-N-acetyl-methionine to free non-α-N-acetylated methionine ratio of at least about 20:1;
b) a free α-N-acetyl-lysine to free non-α-N-acetylated lysine ratio of at least about 20:1;
c) a free α-N-acetyl-threonine to free non-α-N-acetylated threonine ratio of at least about 10:1;
d) a free α-N-acetyl-tryptophan to free non-α-N-acetylated tryptophan ratio of at least about 1.2:1; or
e) a free α-N-acetyl-cysteine to free non-α-N-acetylated cysteine ratio of at least about 100:1.

16. A transgenic seed or transgenic grain produced by the plant of claim 9 having stably incorporated into its genome said polynucleotide.

17. A method of increasing the nutritional value of a seed or a grain comprising
a) stably introducing into the genome of a plant or plant part at least one polynucleotide encoding an amino acid-α-N-acetyltransferase, said polynucleotide is operably linked to a promoter active in the seed of said plant;
b) expressing said polynucleotide in said seed at a sufficient level to allow
i) an increased level of free α-N-acetyl-methionine or free α-N-acetyl-lysine when compared to a control plant not expressing said heterologous polynucleotide;
ii) a free α-N-acetylated amino acid content of at least 220 ppm; or,
iii) a ratio of free α-N-acetylated amino acid content to free non-α-N-acetylated amino acid content of about 1:1.

18. A method of increasing the nutritional value of a seed or a grain comprising stably introducing into the genome of a plant or plant part a first heterologous polynucleotide encoding a first amino acid-α-N-acetyltransferase polypeptide operably linked to a first promoter active in the seed of said plant and a second heterologous polynucleotide encoding a second amino acid-α-N-acetyltransferase polypeptide operably linked to a second promoter active in the seed of said plant, wherein said first and said second amino acid-α-N-acetyltransferase polypeptide acetylate the α-amine of distinct amino acids.

19. The method of claim 18, wherein at least one of said first or said second amino acid-α-N-acetyltransferase polypeptides acetylates the α-amine of at least one of methionine, lysine, threonine, cysteine, or, tryptophan.

20. The method of claim 17, wherein said first or said second promoter comprises a seed-preferred promoter.

21. The method of claim 17, wherein the seed of said plant comprises
a) a free α-N-acetyl-methionine to free non-α-N-acetylated methionine ratio of at least about 20:1;
b) a free α-N-acetyl-lysine to free non-α-N-acetylated lysine ratio of at least about 20:1;
c) a free α-N-acetyl-threonine to free non-α-N-acetylated threonine ratio of at least about 10:1;
d) a free α-N-acetyl-tryptophan to free non-α-N-acetylated tryptophan ratio of at least about 1.2:1; or
e) a free α-N-acetyl-cysteine to free non-α-N-acetylated cysteine ratio of at least about 100:1.

22. The method of claim 17, wherein said free α-N-acetylated amino acid content comprises
a) at least 100 ppm of free α-N-acetyl-methionine;
b) at least 100 ppm of free α-N-acetyl-lysine;
c) at least 1000 ppm of free α-N-acetyl-threonine;
d) at least 100 ppm of free α-N-acetyl-tryptophan; or,
e) at least 100 ppm of free α-N-acetyl-cysteine.
